(19) United States
(12) Patent Application Publication
(10) Pub. No.: US 2015/0182443 A1
(43) Pub. Date: Jul. 2, 2015

(54) TRADITIONAL CHINESE MEDICINE EXTRACT COMPOSITION WITH WHITENING SKIN-CARE FUNCTION ADN ITS PREPARATION METHOD

(71) Applicant: Yuan Yuan, Beijing (CN)

(72) Inventor: Yuan Yuan, Beijing (CN)

(73) Assignee: Shang Chen Mei Bang (Beijing) Biotechnology Co., Ltd., Beijing (CN)

(21) Appl. No.: 14/144,559

(22) Filed: Dec. 31, 2013

(51) Int. Cl. A61K 8/97; A61K 8/73; A61K 8/60; A61Q 19/02; A61K 8/67

(52) U.S. Cl. CPC A61K 8/97; A61Q 19/02; A61K 8/676; A61K 8/60; A61K 8/73

(57) ABSTRACT

The invention provides a raised white skin rejuvenation medicine composition, wherein said composition comprises the following servings by weight of the composition ratio of raw material: 5 to 10 servings of water, 2 to 5 servings of orchids extract, 2 to 3 servings of winter worm of summer, 2 servings of vitamin-C1, 5 servings of almond oil, 2-3 servings of sucrose laurate, 2-3 servings of oat amino acids, 3-4 servings of hyaluronic acid, 2 to 3 servings of Muti pearl, 2 to 3 of Pennisetum extract, 2 to 3 servings of white chrysanthemum extract, 2 to 3 servings of white peony extract, 2 to 3 servings of Poria extract. The above composition was prepared according to the method of the invention, the composition has activated, whitening, moisturizing super protection function can inhibit the formation of melanin and plant extracts such as angelica restore melanin already formed, the epidermal melanin exclusion dilute Huang, make skin white and transparent.

### CROSS-REFERENCE TO RELATED APPLICATIONS

This application claims the priority benefit of Chinese Patent Application Serial No. 201310703312.4, filed on Dec. 19, 2013; Chinese Patent Application Serial No. 201310685138.5, filed on Dec. 13, 2013; Chinese Patent Application Serial No. 201310687223.5, filed on Dec. 13, 2013; Chinese Patent Application Serial No. 201310685115.4, filed on Dec. 13, 2013; Chinese Patent Application Serial No. 201310684837.8, filed on Dec. 13, 2013; Chinese Patent Application Serial No. 201310685112.0, filed on Dec. 13, 2013; Chinese Patent Application Serial No. 201310686861.5, filed on Dec. 13, 2013; Chinese Patent Application Serial No. 201310687218.4, filed on Dec. 13, 2013; Chinese Patent Application Serial No. 201310686865.3, filed on Dec. 13, 2013; Chinese Patent Application Serial No. 201310687250.2, filed on Dec. 13, 2013; Chinese Patent Application Serial No. 201310684846.7, filed on Dec. 13, 2013; Chinese Patent Application Serial No. 201310685166.7, filed on Dec. 13, 2013; and Chinese Patent Application Serial No. 201310687246.6, filed on Dec. 16, 2013, the disclosure of all of which are hereby incorporated by reference in their entirety.

### FIELD OF THE INVENTION

The invention belongs to the field of traditional Chinese medicine, in particular to Chinese medicine extract composition with whitening skin care function and its preparation method.

### BACKGROUND OF THE INVENTION

At present, our management and technical whitening cosmetics there is a big flaw, very serious violations added, some even to the amino mercuric chloride and other contraband as whitening agents such as hydrogen peroxide or peroxide bleach added over the years resulting in a Many beauty accidents, even disfigured, to consumers caused great harm, and has attracted national attention of consumers. In recent years, the whitening agent used in cosmetics mainly compound hydroquinone (hydroquinone), L-ascorbic acid (vitamin C), kojic acid, superoxide dismutase (SOD) and arbutin. However, there are many of these defects whitening agents, such as hydroquinone irritant very large, very unstable vitamin C and SOD, kojic acid also has some irritating and easy discoloration, arbutin role of single and poor effects. Therefore, the development has good security, stability, suppression effect is obvious, a good cost-effective natural whitening agent, has become one of the main research directions pharmaceutical and cosmetic industries, has a very good development prospects. Natural substances used in cosmetic skin treatments and skin care can achieve the double effect, but also versatile, widely applicable, and security features, thus increasing attention.

CN102225039A invention patent published licorice, moutan, Tribulus terrestris, Huang Jing containing motherwort whitening medicine composition for the Chinese medicine composition was prepared using boiling extraction method. Published patent CN101612105 skin whitening medicine composition and its preparation method of Baiji, grabbing, Morus alba, Poria, Tribulus terrestris, Atractylodes, white cardamom, white peony root, angelica Pyrolae and extract composition The compositions were prepared by the method of extraction and separation and enrichment resin soaked.

CN101732223A invention discloses a patent from the aloe vera, white lilac, white cows, Poria, white nuts, Baiji, Angelica, Atractylodes, bird's nest, barley, gentian, angelica, ginkgo composed of pure herbal whitening lotion, the medicine The composition was prepared using the boiling method of extraction.

CN101804185A invention discloses a patent containing Radix, angelica, white peony root, dogwood, Atractylodes, Poria, Chuanxiong, peach, white silkworm, simmer ginger, mint and licorice freckle medicine composition for the use of the use of Chinese medicine composition supercritical carbon dioxide extraction technology to extract.

### SUMMARY OF THE INVENTION

Activation with the present invention, whitening, super moisturizing protective function, can inhibit the formation of melanin and Angelica and other plant extracts to restore melanin is formed, the epidermal melanin excluded, dilute yellowish, make skin white and transparent.

Techniques of the present invention are: the breeding of white skin rejuvenation medicine composition, characterized in that the active ingredient composition made according to the following servings by weight: 5-10 servings water, ylang ylang extract 2-5 servings, servings 2-3 Cordyceps, vitamin-C1-2 servings, 5 servings of almond oil, 2-3 servings of sucrose laurate, 2-3 servings oat amino acids, 3-4 servings by weight hyaluronic acid pearl Muti extract 2-3 servings, three Pennisetum extract 2-3 servings, 2-3 servings of white chrysanthemum extract, 2-3 servings of white peony extract, Poria extract 2-3 servings. Wherein each component may be: 5-6 servings of water, ylang extract was 2-3 servings, Cordyceps 2-3 servings, servings of Vitamin-C 1-2, 2-4 servings of almond oil, sucrose laurate ester 2 -3 servings, oat amino acids 2-3 servings, low molecular weight hyaluronic acid 3-4, 2-3 servings extract pearls Muti, three Pennisetum extract 2-3 servings, 2-3 servings of white chrysanthemum extract, 2-3 servings of white peony extract, Poria extract 2-3 servings.

Wherein each component can be: 8-10 servings of water, ylang extract was 4-5 servings Cordyceps 2-3 servings, servings of Vitamin-C1-2, 4-5 servings of almond oil, sucrose laurate ester 2 -3 servings, oat amino acids 2-3 servings, low molecular weight hyaluronic acid 3-4, 2-3 servings extract pearls Muti, three Pennisetum extract 2-3 servings, 2-3 servings of white chrysanthemum extract, 2-3 servings of white peony extract, Poria extract 2-3 servings.

The preparation method of Chinese medicine composition is: will the medicine after crushing, screening, together with the extraction solvent soak 1˜2 h after extraction machine with a high-pressure homogenizer extraction, the extract was centrifuged using macroporous, with 10 to 95% ethanol elution times, the eluate was concentrated under reduced pressure to extract shape, that was; the extraction solvent is water, ethanol or aqueous ethanol.

The components are extracted through conventional methods together with accessories made cleanser.

Also provides the use of Chinese medicine composition of the above, it applies to the morning after water emulsion or muscle cream, facial volume up and down about the size of each pearl extend evenly onto the skin.

Pharmacological effects:

Yilan: (scientific name: Cananga odorata, Alias: perfume tree, ylang ylang, Latin name: Cananga odorata) is a hard-core flower Annonaceae genus. Native to Southeast Asia, Java, Malaysia, the Philippines. Distributed in Indonesia, the Philippines, Taiwan, Myanmar, Malaysia and mainland China, Guangdong, Sichuan, Yunnan, Fujian, Guangxi and other places, the general was born in the tropics, there has been the introduction of artificial cultivation. Its flavor can be extracted from plants, is the world famous “Yee Lan ” sesame oil, can produce fine fragrances.

Cordyceps sinensis is a parasitic ergot fungus Cordyceps sinensis on Hepialidae insect larvae and larvae of the child seat body complex, which is the culmination of the grass Cordyceps Cordyceps tablets to take the essence of death, there is regulation of the immune system function, anti-tumor, anti-fatigue and other effects, and its cutting-edge theory, a unique formula, exclusive positioning walk in the front end. Vitamin C: also known as L-ascorbic acid, is a water soluble vitamin. Vitamin C in food is absorbed upper small intestine. Once absorbed, it is distributed to all of the water-soluble body structure, normal adult metabolic activity of the body's vitamin C pool of about 1500 mg of vitamin C, the highest peak storage 3000 mg vitamin C.

Almond oil: yellowish transparent and smells, not only is an excellent cooking oil, or an advanced lubricants can withstand low temperatures below −20° C., can be used as important raw material for advanced paint, cosmetics and high-quality soap, also extract flavor and vitamins.

Low molecular weight hyaluronic acid: It is an inherent component of human body, is a dextran aldehyde acid, no species specificity is widely found in the placenta, amniotic fluid, lens, articular cartilage, skin, the dermis and other tissues; organs it is distributed between the cytoplasm and cell stroma, of which contained its own cells and organs from lubrication and nourishment, while providing cellular metabolism microenvironment.

Pearl Farm: Powder molluscs nacre, with calming liver yang; sedative given shock; Liver eyesight.

Lizardtail: As the leaves like mustard leaf, and because the growth in the water, also known as water betel leaves.

White peony: also known as white peony, Paeonia genus is Ranunculaceae. Perennial herbs or subshrubs, underground block or thick; Leaves basal or cauline, large, alternate, pinnate or three fronds or parted; spend big and beautiful, solitary or sometimes bunched top branches, white; sepals 5, persistent; petals 5-10, but often in cultivated species of petals; stamens numerous; disk annular or cup; carpels 2-5, centrifuge development, results into follicles, each there are a number of pieces of seed.

Poria, formerly known as sarsaparilla, aka: Diamond pocket, Diamond thorns, etc., are lily head, Liliaceae climbing shrub , 1-3 m long stems, a few up to 5 m, sparsely spines, petiole 5-15 mm, green, yellow flower, berry 6-15 mm in diameter, red when ripe, there Creme. Flowering from February to May, fruiting from September to November.

After the composition of the present invention, the above ingredients, a synergistic effect of its efficacy.

The beneficial effects of the invention: Herbal extract composition of the invention inhibit melanoma cells, inhibition of tyrosinase, anti-free radical and UV effect, can effectively inhibit skin melanin production, with excellent whitening effect. The composition does not contain synthetic substances, toxic side effects, skin irritation, stable, safe and reliable. In addition to whitening effect, since the composition contains a variety of active ingredients of traditional Chinese medicine, has the function of promoting blood circulation can be provided directly to the skin and nourish their normal physiological process of nutritional ingredients to promote skin metabolism, improve skin health, with good skin care results.

### DETAILED DESCRIPTION OF EMBODIMENTS

### Example 1

A raised white rejuvenation of Chinese medicine composition, characterized in that the active ingredient composition made according to the following servings by weight: 5-6 servings water, ylang ylang extract 2-3 servings, winter worm summer 2-3 servings, servings of Vitamin-C1-2, 2-4 servings of almond oil, 2-3 servings of sucrose laurate, 2-3 servings oat amino acids, 3-4 servings by weight hyaluronic acid, Pearl Extract Muti 2-3 servings, three copies of Pennisetum extract 2-3, 2-3 servings of white chrysanthemum extract, 2-3 servings of white peony extract, Poria extract 2-3 servings.

### Example 2

A raised white rejuvenation of Chinese medicine composition, characterized in that the active ingredient composition made according to the following servings by weight: 8-10 servings water, ylang ylang extract 4-5 copies, winter worm summer 2-3 servings, servings of Vitamin-C1-2, 4-5 servings of almond oil, 2-3 servings of sucrose laurate, 2-3 servings oat amino acids, 3-4 servings by weight hyaluronic acid, Pearl Extract Muti 2-3 servings, three copies of Pennisetum extract 2-3, 2-3 servings of white chrysanthemum extract, 2-3 servings of white peony extract, Poria extract 2-3 servings.

Usage: Apply lotion or water in the morning after muscle cream, facial volume up and down about the size of each pearl extend evenly onto the skin.

Clinical trials:

Grouping: Select age 15 to 60 years old volunteer, volunteers 50 male, 50 female volunteers. The main symptoms: freckles, melasma, sun spots, dull skin. Volunteers were randomly divided into two groups of 50 people per group.

Experimental groups: daily cleansing, on the implementation of facial skin whitening cream coated amount of the present invention prepared in Example 8, morning and evening. Control group: After daily cleansing, the skin on the face amount of the coating commercially available whitening essence, morning and evening. 3 days, 7 days, 1 month, 3 months each time statistics.

Evaluation criteria: by patient reports feeling, and computer than on skin whitening skin texture map to identify the extent and degree of pigmentation fade. Effective: brighten skin tone, pigmentation Color Dodge. Invalid: skin color and pigmentation and experiment before without any change.

Results: 3 days This product was superior to commercially available products with whitening effect, efficiency higher than 80%. 30 days after the effective use of 100%, and also significantly increase the gloss of the skin, the skin delicate mellow. No case of allergic reactions, skin irritation and so on.

The following referenced cases are herein incorporated: U.S. Nonprovisional Patent Application No. 14/______, filed Dec. 30, 2013 with Attorney Docket No. ShangChen-13-01, and entitled “A traditional Chinese medicine composition that has the effect of soothing and cleaning face”; U.S. Nonprovisional Patent Application No. 14/______, filed Dec. 30, 2013 with Attorney Docket No. Shangchen-13-03, and entitled “A traditional Chinese medicine composition for pigment repair”; U.S. Nonprovisional Patent Application No. 14/______, filed Dec. 30, 2013 with Attorney Docket No. Shangchen-13-04, and entitled “A traditional Chinese medicine composition for repair of acne”; U.S. Nonprovisional Patent Application No. 14/______, filed Dec. 30, 2013 with Attorney Docket No. Shangchen-13-05, and entitled “A traditional Chinese medicine composition for anti-wrinkle”; U.S. Nonprovisional Patent Application No. 14/______, filed Dec. 30, 2013 with Attorney Docket No. Shangchen-13-06, and entitled “A traditional Chinese medicine composition for dispelling wrinkles and black eye and its preparation method”; U.S. Nonprovisional Patent Application No. 14/______, filed Dec. 30, 2013 with Attorney Docket No. Shangchen-13-07, and entitled “A Chinese medicine composition for nourishing body care and its preparation method”; U.S. Nonprovisional Patent Application No. 14/______, filed Dec. 30, 2013 with Attorney Docket No. Shangchen-13-08, and entitled “Chinese medicine composition that having acne cleansing effect”; U.S. Nonprovisional Patent Application No. 14/______, filed Dec. 30, 2013 with Attorney Docket No. Shangchen-13-09, and entitled “An anti-aging skin rejuvenation medicine composition and its preparation method”; U.S. Nonprovisional Patent Application No. 14/______, filed Dec. 30, 2013 with Attorney Docket No. Shangchen-13-10, and entitled “Chinese medicine composition for pigment desalination”; U.S. Nonprovisional Patent Application No. 14/______, filed Dec. 30, 2013 with Attorney Docket No. Shangchen-13-011, and entitled “Chinese medicine composition for acne scar repair”; U.S. Nonprovisional Patent Application No. 14/______, filed Dec. 30, 2013 with Attorney Docket No. Shangchen-13-012, and entitled “Chinese medicine composition for Prevent from aging and soothing the skin”; and U.S. Nonprovisional Patent Application No. 14/______, filed Dec. 30, 2013 with Attorney Docket No. Shangchen-13-013, and entitled “Chinese medicine composition for Nourishing the skin and its preparation method”, the disclosure of all of which are herein incorporated by reference in its entirety.

what is claimed is:

1. A whitening rejuvenation Chinese medicine composition, characterized in that the active ingredient composition made according to the following servings by weight: 5-10 servings water to extract composition according orchids 2-5 servings, winter worm summer 2 -3 servings, servings of Vitamin-C1-2, 2-5 servings of almond oil, 2-3 servings of sucrose laurate, 2-3 servings oat amino acids, 3-4 servings by weight hyaluronic acid, Pearl Extract 2 Muti-3 copies, three copies of Pennisetum extract 2-3, 2-3 servings of white chrysanthemum extract, 2-3 servings of white peony extract, Poria extract 2-3 servings.

2. The Chinese medicine composition as claimed in claim 1, wherein the components are: 5-6 servings of water, ylang extract was 2-3 servings, Cordyceps 2-3 servings, servings of Vitamin-C1-2, almond 2-4 servings oil, sucrose laurate 2-3 servings, 2-3 servings of oat amino acids, low molecular weight hyaluronic acid 3-4, 2-3 servings of pearl Muti extract, extract 2-3 Saururi copies, white chrysanthemum extract 2-3 servings, 2-3 servings of white peony extract, Poria extract 2-3 servings.

3. The Chinese medicine composition as claimed in claim 1, wherein, wherein the components are: 8-10 servings water, ylang extract was 4-5 servings, servings 2-3 Cordyceps, vitamin-C1-2 servings of almond 4-5 servings of oil, sucrose laurate 2-3 servings, 2-3 servings of oat amino acids, low molecular weight hyaluronic acid 3-4, 2-3 servings of pearl Muti extract, extract 2-3 Saururi copies, white chrysanthemum extract 2-3 servings, 2-3 servings of white peony extract, Poria extract 2-3 servings.

4. The Chinese medicine composition as claimed in claim 1, obtained using high pressure homogenization extraction: the medicine after the crushing, screening, and extraction with a solvent soaked 1-2 h, the extraction machine with a high pressure homogenizer, the extraction solution after centrifugation with macroporous resin, and then 10 to 95% ethanol several times eluent is collected, evaporated to extract like that too; said extraction solvent is water, ethanol, or aqueous ethanol.

5. The composition of claim 1, wherein the herbal extracts used in the preparation of cosmetic skin whitening skin requirements.

6. Cleanser medicine composition as described in claim is made, it applies to the morning after water emulsion or muscle cream, facial volume up and down about the size of each pearl extend evenly onto the skin.
